(19) United States
(12) Patent Application Publication
(10) Pub. No.: US 2025/0302608 A1
(43) Pub. Date: Oct. 2, 2025

(54) ACCOMMODATING INTRAOCULAR LENS (AIOL) DEVICE

(71) Applicant: Adaptilens, Inc., Chestnut Hill, MA (US)

(72) Inventors: Liane CLAMEN, Chestnut Hill, MA (US); Dan STANLEY, Chestnut Hill, MA (US); Brett THOMES, Chestnut Hill, MA (US)

(73) Assignee: Adaptilens, Inc., Chestnut Hill, MA (US)

(21) Appl. No.: 19/086,943

(22) Filed: Mar. 21, 2025

Related U.S. Application Data
(60) Provisional application No. 63/568,331, filed on Mar. 21, 2024.
(60) Provisional application No. 63/568,111, filed on Mar. 21, 2024.

(51) Int. Cl. A61F 2/16

(52) U.S. Cl. CPC A61F 2/1635; A61F 2/164

(57) ABSTRACT

An accommodating intraocular (AIOL) lens is configured to provide an amount of accommodation to allow a continuous range of vision that imitates the healthy human crystalline lens. The AIOL configuration include both filled and unfilled lens bodies with non-uniform external anterior surfaces. In embodiments, the AIOL includes a shell body and a filler material disposed within the shell body, wherein the thickness of either the anterior wall or posterior wall is non-uniform. In other embodiments, the AIOL includes a pliable lens body of an optically clear copolymer defined by an anterior surface and a posterior surface, wherein one of the anterior surface or posterior surfaces characterized by a discontinuity in the optical surface.

### CROSS REFERENCE TO RELATED APPLICATION

This application claims the benefit of priority to the following applications, the entire contents of all of which are incorporated herein by this reference for all purposes:
U.S. Provisional Application Ser. No. 63/568,111, entitled “DESIGN OF AN ACCOMMODATING INTRAOCULAR LENS (AIOL)” filed on Mar. 21, 2024, and,
U.S. Provisional Application Ser. No. 63/568,331, entitled “DESIGN OF AN ACCOMMODATING INTRAOCULAR LENS (AIOL)” filed on Mar. 21, 2024.

### FIELD OF THE INVENTION

The disclosure relates to intraocular lenses and, more particularly, to various embodiments of accommodating intraocular lens (AIOL).

### BACKGROUND OF THE INVENTION

The lens capsule, or the capsular bag, of the eye is a thin membrane around the eye's natural lens that holds the lens in a central position within the eye and helps give the lens its shape. The capsular bag comprises an anterior and posterior capsule. Attached to the periphery of the capsule are tiny string-like structures called zonules which attach to the ciliary muscle. The ciliary muscle plays a critical role in accommodation, the process that allows the eye to adjust focus for near, intermediate, and distance vision. For near vision, the ciliary muscle contracts and reduces the tension on the zonules. This allows the lens to become rounder, thus increasing its refractive power so the eye can focus on near objects. For distance vision, the ciliary muscle relaxes, increasing tension on the zonules and pulling the capsular bag taut. Thus, the lens flattens and has decreased refractive power, enabling the eye to focus on distant objects.

As people reach about age 45 and older, the lens becomes stiffer and less able to change shape and power. With this age-related condition, called presbyopia, people must use glasses or contact lenses to see clearly at near and intermediate. At about the age of 75, the natural lens becomes stiffer and cloudy, a condition known as a cataract. A cataract can be removed with cataract surgery during which an artificial intraocular lens or IOL is inserted into the lens capsule in place of the cloudy cataractous lens.

The purpose of an accommodating intraocular lens (AIOL) is to restore the ability of the human eye to accommodate under typical visual stimuli. An accommodating intraocular lens works with the ciliary muscles to allow people to see over a range of distances. The AIOL is surgically inserted into the capsular bag of the eye during cataract surgery, after the cataractous lens is removed. An AIOL may be made of material(s) that imitate the optical and mechanical properties of a healthy, young lens.

An AIOL may comprise a lens body in the form of a hollow shell which is expanded with a filler material. Examples of this type of AIOL are disclosed in Applicant's prior U.S. Pat. Nos. 10,278,810 and 11,678,976 which both describe fluid filled, accommodating IOLs comprising a capsular shell or interface enclosing an optically acceptable medium. The medium provides shape to the capsular interface, optical power, and a physiologic response to the suspensory ligament.

More recently an “all-in-one” AIOL design comprising a soft, flexible material that does not require in situ filling is being developed. International Publication WO2023225332A1 describes polymers that can be used to make an all-in-one AIOL that does not require a shell. International Publication WO2024233709A2 also describes an optically clear bottlebrush copolymer that can be used to make an all-in-one AIOL that does not require a shell.

However, published accommodating IOL designs, in general, do not provide details related to maximizing the accommodative amplitude and overall optical performance by specifically designing different portions of the lens with varying criteria.

Accordingly a need exists for an accommodating intraocular lens that maximizes the accommodative amplitude of the lens.

Another need exists for an accommodating intraocular lens that maximizes overall optical performance of the lens.

A further need exists for an accommodating intraocular lens in which different portions of the lens are designed according to specific performance criteria to maximize the accommodative amplitude and/or overall optical performance of the lens.

### SUMMARY OF THE INVENTION

The disclosed accommodating intraocular lens is configured to provide an amount of accommodation to allow a continuous range of vision that imitates the healthy human crystalline lens. The AIOL configurations include both filled and unfilled lens bodies with non-uniform external anterior surfaces. In embodiments, the AIOL includes a shell body and a filler material disposed within the shell body, wherein the thickness of either the anterior wall or posterior wall of the shell body is non-uniform. In other embodiments, the AIOL includes a pliable lens body of an optically clear material wherein one of the exterior anterior surface or posterior surfaces characterized by a discontinuity in curvature.

According to one aspect of the disclosure, an accommodating intraocular lens comprises: a shell body having an anterior wall and a posterior wall bounded by a perimeter to define an interior volume therebetween; and a filling material disposed within the interior volume, wherein each of the anterior wall and posterior wall have a thickness dimension measurable between respective exterior and interior surfaces thereof, and wherein the thickness dimension of at least one of the anterior wall and the posterior wall is non-uniform within the perimeter of the lens body. In embodiments, the thickness dimension of the posterior wall is non-uniform within the perimeter of the lens body. In embodiments, the thickness dimension of the posterior wall is different at a posterior pole of the shell body than a thickness dimension of the posterior wall at the perimeter. In embodiments, the thickness dimension of the posterior wall is greater at the posterior pole of the shell body than the thickness dimension of the posterior wall at the perimeter. In embodiments, the thickness dimension of the posterior wall is lesser at the posterior pole of the shell body than the thickness dimension of the posterior wall at the perimeter. In embodiments, the thickness dimension of the anterior wall is non-uniform within the perimeter of the lens body. In embodiments, the thickness dimension of the anterior wall is different at an anterior pole of the shell body than the thickness dimension of the anterior wall at the perimeter. In embodiments, the thickness dimension of the anterior wall is greater at the anterior pole of the shell body than the thickness dimension of the anterior wall at the perimeter. In embodiments, the thickness dimension of the anterior wall is lesser at the anterior pole of the shell body than the thickness dimension of the anterior wall at the perimeter. In embodiments, the shell comprises at least one of a silicone or an acrylate. In embodiments, the filling material comprises poly(dimethylsiloxane) and a functionalized monomer. In embodiments, the filling material comprises one or more monomethacryloxy terminated polydimethylsiloxane (PDMS-MA) units and one or more monomethacryloxy terminated fluorinated siloxane (Fluoro-MA) units. In embodiments, the filler material has a specific gravity of between approximately 0.9 and 1.4. In embodiments, the thickness of the anterior wall is between approximately 0.05 mm and 1.0 mm. In embodiments, the thickness of the posterior wall is between approximately 0.05 mm and 1.0 mm. In embodiments, the radius of the posterior wall interior surface is between approximately 2.0 to 20.0 mm. In embodiments, the radius of the posterior wall exterior surface is between approximately 2.0 mm to 20.0 mm. In embodiments, the radius of the anterior wall interior surface is between approximately 2.0 mm to 20.0 mm. In embodiments, the radius of the anterior wall exterior surface is between approximately 2.0 mm to 20.0 mm. In embodiments, one of the anterior wall and a posterior wall comprise a material having a refractive index between approximately 1.400 and 1.600. In embodiments, one of the anterior wall and posterior wall comprise material having a shell modulus between 25 and 31 psi. In embodiments, the filler material has a refractive index of between approximately 1.350 and 1.550. In embodiments, the filler has material has a refractive index of between approximately 1.400 and 1.430. In embodiments, the filler material has a viscosity between 1 Pa·s and 20 Pa·s. In embodiments, the filler has material has a viscosity of between 1 Pa·s and 10 Pa·s.

According to another aspect of the disclosure, an accommodating intraocular lens comprises: a pliable lens body having an interior volume defined by an interior anterior surface and an interior posterior surface bounded by a perimeter, wherein one of the interior anterior surface and the interior posterior surface is characterized by a discontinuity in curvature within the perimeter of the lens body. In embodiments, the interior posterior surface is characterized by an abrupt change in curvature within the interior volume. In embodiments, the abrupt change in curvature of the interior posterior surface comprises an increase of greater than approximately 5%. In embodiments, the abrupt change in curvature of the interior posterior surface comprises a decrease of greater than approximately 5%. In embodiments, the interior anterior surface is characterized by an abrupt change in curvature within the interior volume. In embodiments, the abrupt change in curvature of the interior anterior surface comprises an increase of greater than approximately 5%. In embodiments, the abrupt change in curvature of the interior anterior surface comprises a decrease of greater than approximately 5%. In embodiments, the discontinuity in curvature within the interior volume of the lens body comprises a transition from a spherical interior posterior surface to a non-spherical interior posterior surface. In embodiments, the discontinuity in curvature within the interior volume of the lens body comprises a transition from a spherical interior anterior surface to a non-spherical interior anterior surface.

According to yet another aspect of the disclosure, an accommodating intraocular lens comprises: a pliable lens body having an exterior anterior surface and an exterior posterior surface bounded by a perimeter, wherein one of the interior anterior surface and interior posterior surface is characterized by a discontinuity in curvature within the perimeter of the lens body.

According to yet another aspect of the disclosure, an accommodating intraocular lens comprises: a pliable accommodating lens body having a curved exterior anterior surface and a curved exterior posterior surface bounded by a perimeter, wherein less than all of the exterior anterior surface or the exterior posterior surface has a common curvature.

According to yet another aspect of the disclosure, an accommodating intraocular lens comprises: a pliable lens body having an exterior anterior surface and an exterior posterior surface bounded by a perimeter, wherein one of the exterior anterior surface and exterior posterior surface is characterized by a discontinuity in curvature within the perimeter of the lens body. In embodiments, the discontinuity in curvature within the perimeter of the lens body comprises a transition from a spherical exterior posterior surface to a non-spherical exterior posterior surface. In embodiments, the discontinuity in curvature within the perimeter of the lens body comprises a transition from a spherical exterior anterior surface to a non-spherical exterior anterior surface.

According to yet another aspect of the disclosure, an accommodating intraocular lens comprises: a pliable accommodating lens body having an exterior anterior surface and an exterior posterior surface bounded by a perimeter, wherein a central region of the exterior anterior surface within the perimeter comprises a spherical lens portion surrounded by an aspheric lens portion. In embodiments, approximately 45% to 55% of the exterior anterior surface within the perimeter comprises an aspheric lens.

According to yet another aspect of the disclosure, an accommodating intraocular lens comprises: an aspheric lens body defined by an exterior anterior surface and an exterior posterior surface bounded by a perimeter, the lens body comprising a material capable of accommodation of the lens body in response to ciliary muscle stimulus without further structure coupled to the perimeter of the lens body.

According to yet another aspect of the disclosure, an accommodating intraocular lens comprises: a pliable lens body comprising an optically clear material, the lens body defining an exterior anterior surface and an exterior posterior surface bounded by a perimeter, wherein one of the exterior anterior surface and the exterior posterior surface is characterized by a discontinuity in radius within the perimeter of the lens body. In embodiments, the optically clear material comprises either bottle brush polymer or a copolymer. In embodiments, the optically clear material comprises a bottle brush polymer or bottle brush copolymer. In embodiments, the abrupt change in radius of the exterior anterior surface comprises a decrease in radius of greater than approximately 5%. In embodiments, the abrupt change in radius of the exterior anterior surface comprises an increase in radius of greater than approximately 5%.

According to yet another aspect of the disclosure, an accommodating intraocular lens comprises: a pliable lens body comprising an optically clear material, the lens body defining an exterior anterior surface and an exterior posterior surface bounded by a perimeter, wherein one of the exterior anterior surface and the exterior posterior surface is characterized by a discontinuity in curvature within the perimeter of the lens body.

According to yet another aspect of the disclosure, an accommodating intraocular lens comprises: a shell, the shell having a modulus and a refractive index; and a filler material disposed within the shell, the filler having a viscosity and a refractive index, wherein the intraocular lens is configured to provide an amount of accommodation to allow a continuous range of vision.

According to yet another aspect of the disclosure, an accommodating intraocular lens comprises: a pliable lens body having an exterior anterior surface and an exterior posterior surface bounded by a perimeter, wherein one of the exterior anterior surface and exterior posterior surface is characterized by a discontinuity in curvature within the perimeter of the lens body, and wherein the lens body is configured to provide an amount of accommodation to allow a continuous range of vision.

According to yet another aspect of the disclosure, an accommodating intraocular lens comprises: a pliable lens body having an exterior anterior surface and an exterior posterior surface bounded by a perimeter, wherein one of the exterior anterior surface and exterior posterior surface is characterized by a discontinuity in curvature within the perimeter of the lens body, and wherein the lens body is configured to provide an amount of optical power to allow a continuous range of vision.

According to yet another aspect of the disclosure, an accommodating intraocular lens comprises: a pliable lens body comprising an optically clear material, the lens body defining an exterior anterior surface and an exterior posterior surface bounded by a perimeter, wherein one of the exterior anterior surface and the exterior posterior surface is characterized by a discontinuity in radius within the perimeter of the lens body. In embodiments, the optically clear material comprises an optically clear copolymer. In embodiments, the optically clear material comprises a bottle brush polymer or bottle brush copolymer. In embodiments, the abrupt change in radius of the exterior anterior surface comprises an decrease in radius of greater than approximately 5%. In embodiments, the abrupt change in radius of the exterior anterior surface comprises an increase in radius of greater than approximately 5%.

According to still another aspect of the disclosure, the disclosed AIOL comprises a shell and a filler material disposed within the shell. The shell has a modulus and a refractive index. The filler has a viscosity and a refractive index. In embodiments, the shell may be at least one of a silicone or an acrylate. In embodiments, the material properties of the shell may include: a clear appearance; a refractive index between approximately 1.35 and approximately 1.550; a specific gravity between approximately 0.9 and approximately 1.3; a Shore A hardness of between approximately 6 and approximately 12; a tensile strength of between approximately 750 and approximately 1000 psi; an elongation of between approximately 700 and approximately 1500%; and a modulus of between approximately 10 psi and approximately 50 psi.

The filler may include a poly(dimethylsiloxane) and a functionalized monomer. The filler may have material properties including: a clear appearance; a refractive index between approximately 1.350 and approximately 1.550; a specific gravity of between approximately 0.9 and approximately 1.4; and a viscosity between approximately 1 Pa·s and approximately 20 Pa·s. The amount of accommodation of the accommodating intraocular lens may be described by the thick lens equations disclosed herein.

### BRIEF DESCRIPTION OF THE DRAWINGS

Those skilled in the art should more fully appreciate advantages of various embodiments of the invention from the following “Detailed Description” discussed with reference to the drawings summarized immediately below.

FIG. 1A illustrates conceptually a top view of an accommodating intraocular lens according to embodiments described herein.

FIG. 1B illustrates conceptually a side view of the accommodating intraocular lens of FIG. 1A.

FIG. 1C illustrates conceptually a cross-sectional view of the accommodating intraocular lens of FIG. 1B as viewed along line A-A.

FIG. 2 illustrates conceptually various structural attributes of an accommodating intraocular lens according to embodiments described herein.

FIG. 3 illustrates conceptually various structural attributes of an accommodating intraocular lens according to embodiments described herein.

FIG. 4 illustrates conceptually various structural attributes of an accommodating intraocular lens according to embodiments described herein.

FIG. 5 illustrates conceptually various structural attributes of an accommodating intraocular lens according to embodiments described herein.

FIG. 6 illustrates conceptually an accommodating intraocular lens of FIG. 4 in a near focus state according to embodiments described herein.

FIG. 7 illustrates conceptually an accommodating intraocular lens of FIG. 4 in a distance focus state according to embodiments described herein.

FIG. 8 illustrates conceptually the structural and refractive attributes of solid accommodating intraocular lens according to embodiments described herein.

FIGS. 9A and 9B are top and side cut-away views of an accommodating intraocular lens according to an Example 1 described herein.

FIGS. 10A and 10B are top and side cut-away views of an accommodating intraocular lens according to an Example 2 described herein.

FIG. 10C illustrates conceptually a partial cross-sectional view of the shell wall of the accommodating intraocular lens of FIG. 10B.

FIG. 11A illustrates conceptually a top view of an accommodating intraocular lens according to Example 3 described herein.

FIG. 11B illustrates conceptually a side view of the accommodating intraocular lens of FIG. 11A.

FIGS. 12A-12C are top, side cut-away and perspective views of an accommodating intraocular lens according to an Example 4 described herein.

### DETAILED DESCRIPTION

Disclosed is an accommodating intraocular lens (AIOL) the restores the ability of the human eye to accommodate under typical visual stimuli by materials and geometries that mimic the mechanical and optical properties of the young Human Crystalline Lens (HCL). Two variations of an accommodating intraocular lens (AIOL) are described herein, where one is in the accommodated state at rest and the other is in the unaccommodated state at rest. These AIOLs are intended to change shape and thus change amplification power to provide a continuous range of vision.

In embodiments, an AIOLs have a lens body comprising a shell with a filler, intended for placement in the capsular bag, focusing ciliary body displacements on specific regions of the lens that result in IOL shape changes, thereby: 1) maximizing power change of the AIOL, including: a) changes in the thickness of the AIOL and b) radius changes in the AIOL; 2) providing a stable surface in regions of the AIOL for inclusion of optical enhancement features (e.g., astigmatism correction, etc.); and 3) providing the ability to deliver the lens into the capsular bag through a small incision using typical intraocular lens delivery methods such as existing intraocular lens delivery devices or a novel intraocular lens delivery system, developed for delivery of this AIOL.

As used herein, the disclosed AIOL lens bodies are capable of accommodation in response to ciliary muscle stimulus without further haptic structures coupled exteriorly of the perimeter 25 of the lens body 15, e.g. springs, wings, pontoons, auxiliary reservoirs, or other structures, etc. used to maintain the lens body within the capsular bag.

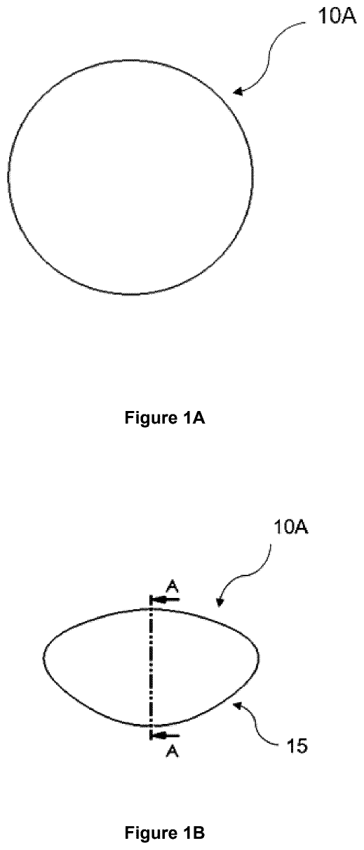

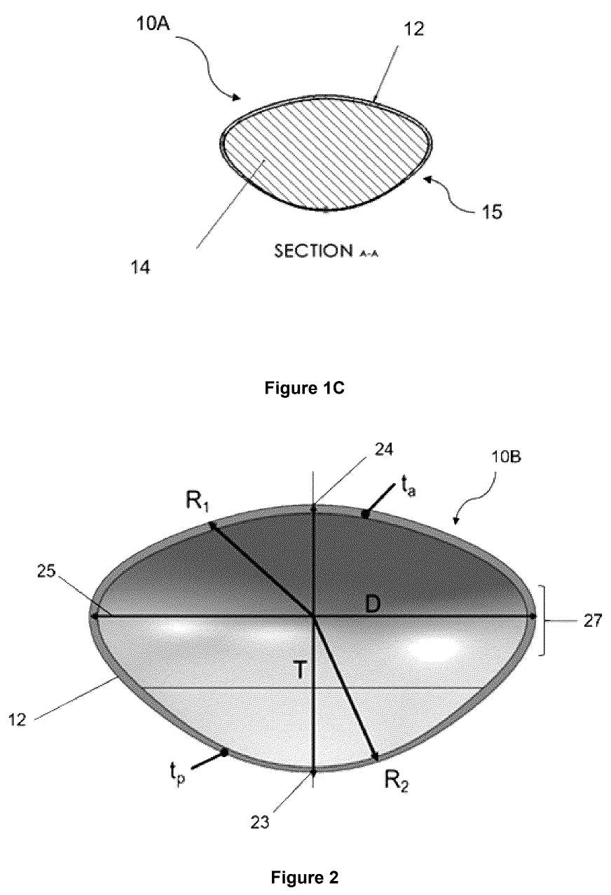

As shown in FIGS. 1A-C, an AIOL 10A has a lens body 15 comprising a shell 12 and a filler 14. The materials of the shell 12 and filler 14 may be selected to closely match the HCL to provide maximal shape and thus power change. The overall dimensions of the AIOL 10A are selected to match the average dimensions of an HCL. In embodiments, the filler 14A may comprise a proprietary bottle-brush polymer (BBP), such as those disclosed in any of US20240287232A1 or International Publication WO2022246198A1 or International Publication WO2024233709A2. The shell 12 may comprise a silicone rubber or acrylate or similar material that provides safety, stability, and with properties within the ranges shown in Table 1.

TABLE 1

| Properties | Typical Value |
|---|---|
| Appearance | Clear |
| Refractive Index | 1.35-1.550 |
| Specific Gravity | 0.9-1.3 |
| Hardness, Shore A | 6-12 |
| Tensile Strength, psi | 750-1000 |
| Elongation, % | 700-1500 |
| Modulus, 100%, psi | 10-50 |

In certain exemplary embodiments, the filler 14 is an optically clear, biostable bottlebrush polymer with mechanical and optical properties that have been fine-tuned to meet the requirements of the AIOL. Bottlebrush polymers comprise many polymer side chains densely grafted to a linear chain (backbone). The high grafting density leads to a steric repulsion between the side chains which forces the polymer backbone to adopt an extended conformation. This extended, wormlike conformation can be controlled by variables such as side-chain length and grafting density without changing the chemical composition. In certain exemplary embodiments, the bottlebrush polymer may comprise poly(dimethylsiloxane) (PDMS) and a functionalized monomer. The bottle brush polymer is designed to produce ultra-high molecular weight components with a large volume architectural cross section that prevents diffusion of the filler through the silicone shell, unlike their linear PDMS counterparts that include low/mid molecular weight components. In bulk bottlebrush melts (without solvent), like in the AIOL, the extended conformation reduces the chain entanglement density of the wormlike molecules resulting in rheological properties with an ultralow plateau modulus of 102 Pa to 103 Pa, which is much lower than the 105 Pa to 106 Pa typically observed in melts of linear polymers.

Typical properties of the filler 14 material are listed in Table 2 which lists various properties of the filler 14 and provides a range of potential values for each property. The formulation allows for precise control of the refractive index of the filler 14, e.g. a bottlebrush polymer, and its mechanical properties required for the filler to fit into the shell, while avoiding the use of solvents or other diluting fluids. The filler 14 is injected into the cured shell to a precise volume to achieve the final shape of the AIOL.

TABLE 2
AIOL Fill Material Properties

| Properties | Typical Value |
|---|---|
| Appearance | Clear |
| Refractive Index | 1.350-1.550 |
| Specific Gravity | 0.9-1.4 |
| Viscosity, Pa · s | 1-20 |

FIG. 2 illustrates an AIOL 10B comprising a shell and a filler similar to that illustrated in FIG. 1. The attributes of AIOL 10B are listed below:
tₐ—Anterior shell thickness
tₚ—Posterior shell thickness
R₁—Anterior radius, outside
R₂—Anterior radius, inside
D—Overall diameter
T—Central thickness
   The attributes of the material of shell 12 include Modulus and Refractive index. The attributes of the material of filler 14 include Viscosity and Refractive index. Table 3 below lists the attributes and corresponding value ranges of the AIOL 10B:

TABLE 3

| Attribute | Range |
|---|---|
| Anterior Shell Thickness (mm), ta | 0.1-0.3 |
| Posterior Shell Thickness (mm), tp | 0.1-0.3 |
| Anterior Radius (mm) R₁ | Varies according to desired lens |
| | power |
| Posterior Radius (mm) R₂ | Varies according to desired lens |
| | power |
| Overall Diameter (mm), D | 9.0-10.0 |
| Central Optic Thickness (mm), T | 4.0-5.0 |
| Shell Modulus (psi) | 10-50 |
| Shell Refractive Index | 1.400-1.430 |
| Filler Viscosity, (Pa · s) | 1-10 |
| Filler Refractive Index | 1.400-1.430 |

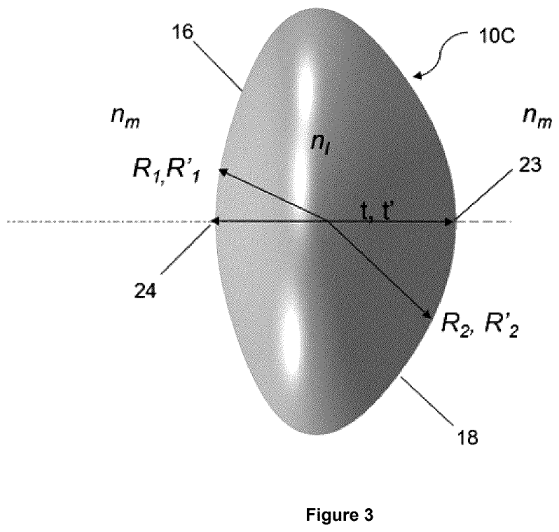

The AIOL 10C of FIG. 3 represent both an AIOL having a lens body comprising a flexible shell with a viscous liquid filler such as AIOL 10B, or a lens body comprising a flexible material such as AIOL 10J described herein. The attributes of AIOL 10C of in FIG. 3, used for calculating an amount of Accommodation, P, are listed below.
nₗ—Lens refractive index
nₘ—Aqueous (eye) refractive index
R₁—Radius of anterior surface, initial state
R′₁—Radius of anterior surface, final state
R₂—Radius of posterior surface, initial state
R′₂—Radius of posterior surface, final state
t—Central lens thickness, initial state
t′—Central lens thickness, final state
f—Focal length, initial state
f′—Focal length, final state
P—Accommodation

The amount of accommodation, P, of the of the AIOL 10C can be estimated using the Thick Lens equation, shown below, including a determination of Initial State and a Final State values.

$$Initial State$$

$$\frac{1}{f} = ( {n}_{l} - {n}_{m} ) [ \frac{1}{{R}_{1}} - \frac{1}{{R}_{2}} + ( {n}_{l} - {n}_{m} ) t / {n}_{l} {R}_{1} {R}_{2} ]$$

$$Final State$$

$$\frac{1}{{f}^{′}} = ( {n}_{l} - {n}_{m} ) [ \frac{1}{{R}_{1}^{′}} - \frac{1}{{R}_{2}^{′}} + ( {n}_{l} - {n}_{m} ) {t}^{′} / {n}_{l} {R}_{1}^{′} {R}_{2}^{′} ]$$

$$P = \frac{1}{f} - \frac{1}{{f}^{′}}$$

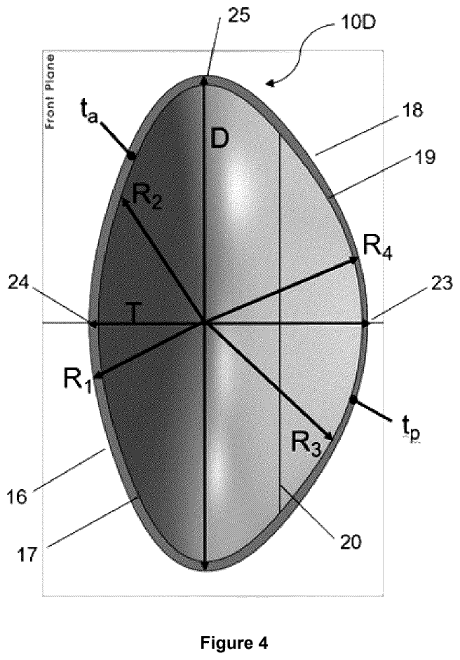

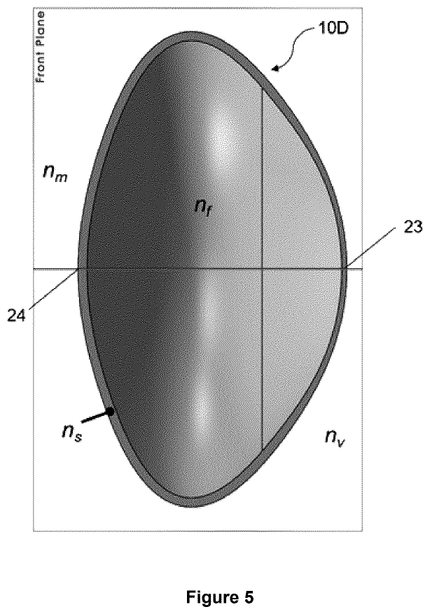

FIGS. 4 and 5 illustrate an AIOL 10D comprising a shell 12 and a filler 14 having the following attributes:
tₐ—Anterior shell thickness
tₚ—Posterior shell thickness
R₁—Anterior radius, outside
R₂—Anterior radius, inside
R₃—Posterior radius, inside
R₄—Posterior radius, outside
D—Overall diameter
T—Central thickness
nₘ—Aqueous (eye) refractive index
nₛ—Lens shell refractive index
n_{f}—Lens filler refractive index
nᵥ—Vitreous humor (eye) refractive index

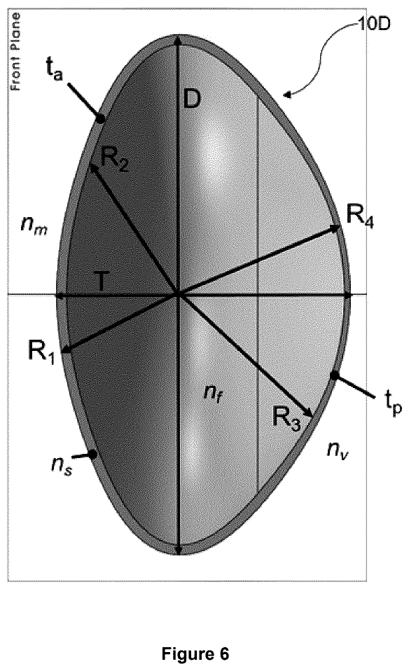

The AIOLs disclosed herein are intended to change shape and thus change power to provide a continuous range of vision. FIG. 6 illustrates conceptually AIOL 10D in a near focus state. In the near focus state, the overall near power, P_{N}, can be calculated using the Equations (1) through (5) as set forth below.

### Lens Power in Near Focus State:

$$Surface 1 : {P}_{1} = ( {n}_{s} - {n}_{m} ) / {R}_{1} \qquad ( 1 )$$

$$Surface 2 : {P}_{2} = ( {n}_{f} - {n}_{s} ) / {R}_{2} \qquad ( 2 )$$

$$Surface 3 : {P}_{3} = ( {n}_{s} - {n}_{f} ) / {R}_{3} \qquad ( 3 )$$

$$Surface 4 : {P}_{4} = ( {n}_{v} - {n}_{s} ) / {R}_{4} \qquad ( 4 )$$

$$Overall power , Near : {P}_{N} = {P}_{1} + {P}_{2} + {P}_{3} + {P}_{4} - ( T / {n}_{f} ) * ( {P}_{1} + {P}_{2} ) * ( {P}_{3} + {P}_{4} ) \qquad ( 5 )$$

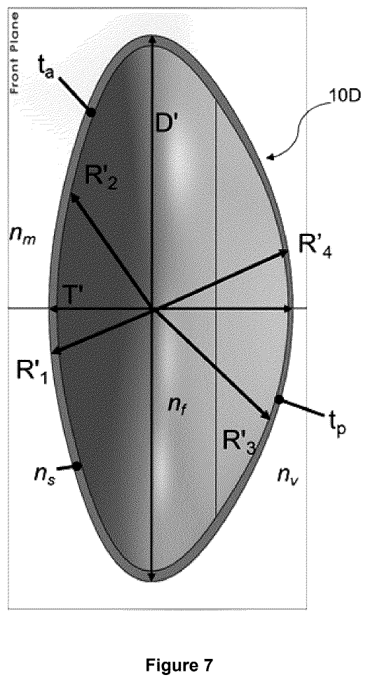

FIG. 7 illustrates conceptually AIOL 10D in a distance focus state. In the near focus state, the overall distance power, P_{D}, can be calculated using the Equations (6) through (10) as set forth below.

### Lens Power in Distance Focus State:

$$Surface 1 : {P}_{1}^{′} = ( {n}_{s} - {n}_{m} ) / {R}_{1}^{′} \qquad ( 6 )$$

$$Surface 2 : {P}_{2}^{′} = ( {n}_{f} - {n}_{s} ) / {R}_{2}^{′} \qquad ( 7 )$$

$$Surface 3 : {P}_{3}^{′} = ( {n}_{s} - {n}_{f} ) / {R}_{3}^{′} \qquad ( 8 )$$

$$Surface 4 : {P}_{4}^{′} = ( {n}_{v} - {n}_{s} ) / {R}_{4}^{′} \qquad ( 9 )$$

$$Overall power , Distance : {P}_{D} = {P}_{1}^{′} + {P}_{2}^{′} + {P}_{3}^{′} + {P}_{4}^{′} - ( {T}^{′} / {n}_{f} ) * ( {P}_{1}^{′} + {P}_{2}^{′} ) * ( {P}_{3}^{′} + {P}_{4}^{′} ) \qquad ( 10 )$$

Given the calculated overall near power, P_{N}, and overall distance power, P_{D}, the Accommodating Power of AIOL 10D can be calculated as follows:

$$Accommodating Power = {P}_{N} - {P}_{D}$$

### Flexible Solid AIOL

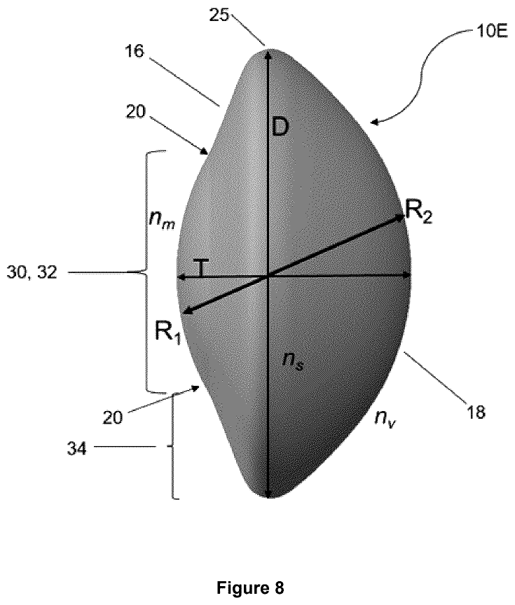

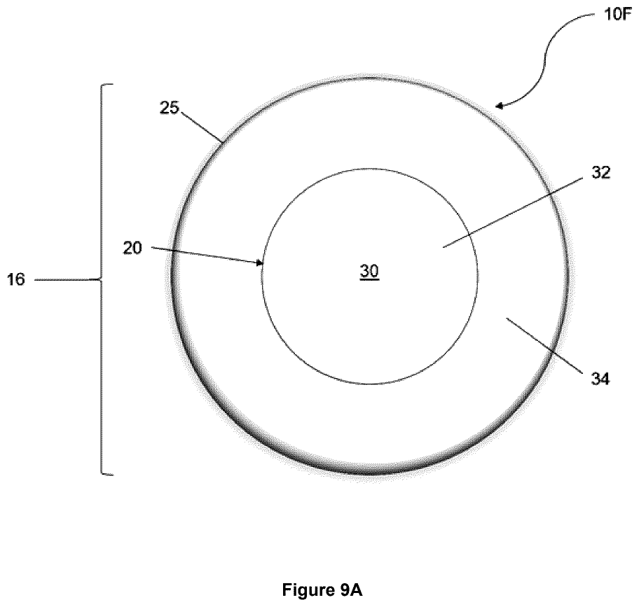

As illustrated in FIG. 8, an AIOL 10E comprises a lens body 15 made of an optically clear material, such as a copolymer or a bottle brush polymer or copolymer. The lens body 15 defines an exterior anterior surface 16 and an exterior posterior surface 18 and perimeter 25 at the equator 27 of the lens body 15. One of the exterior anterior surface 16 or the exterior posterior surface 18 is characterized by an abrupt change or discontinuity 20 of radius and anterior pole 24 of the lens body 15. In this embodiment, a shell, which provides support to the filler material in the other embodiments disclosed herein, is absent. The optically clear copolymer material itself is a self-supported structure. The various attributes of the AIOL 10E are set forth in Table 4.

TABLE 4

| Attribute | Value (mm) |
|---|---|
| R₁ | 5.0 |
| R₂ | 5.0 |
| D | 9.5 |
| T | 4.7 |

Equations (11) through (17) below can be used to calculate the Accommodating Power of AIOL 10E, given the radius values of the anterior surface and posterior surfaces.

### Lens Power in Near Focus State:

$$Surface 1 : {P}_{1} = ( {n}_{s} - {n}_{m} ) / {R}_{1} \qquad ( 11 )$$

$$Surface 2 : {P}_{2} = ( {n}_{v} - {n}_{s} ) / {R}_{2} \qquad ( 12 )$$

$$Overall power , Near : {P}_{N} = {P}_{1} + {P}_{2} - ( T / {n}_{s} ) * ( {P}_{1} + {P}_{2} ) \qquad ( 13 )$$

### Lens Power in Distance Focus State:

$$Surface 1 : {P}^{′} 1 = ( {n}_{s} - {n}_{m} ) / {R}_{1}^{′} \qquad ( 14 )$$

$$Surface 2 : {P}^{′} 2 = ( {n}_{v} - {n}_{s} ) / {R}_{2}^{′} \qquad ( 15 )$$

$$Overall power , Distance : {P}_{D} = {P}_{1}^{′} + {P}_{2}^{′} - ( {T}^{′} / {n}_{s} ) * ( {P}_{1}^{′} + {P}_{2}^{′} ) \qquad ( 16 )$$

$$Accommodating Power = {P}_{N} - {P}_{D} \qquad ( 17 )$$

### Manufacturing Methods

In accordance with another aspect of the disclosure, a method of manufacturing an accommodating intraocular lens (AIOL) includes molding an anterior half shell using an anterior half shell mold having an inside anterior mold half and an outside anterior mold half. The inside anterior mold half has an outside surface being adjacent to an inside surface of the anterior half shell. A first polymer is injected into the anterior half shell mold. The anterior half shell is cured in the anterior half shell mold to form the anterior half shell. The inside anterior mold half is separated from the outside anterior mold half.

The method of manufacturing an AIOL includes molding a posterior half shell using a posterior half shell mold having an inside posterior mold half and an outside posterior mold half. The inside posterior mold half has an outside surface being adjacent to an inside surface of the outside posterior half shell. A second polymer is injected into the posterior half shell mold. The posterior half shell is cured in the posterior half shell mold to form the posterior half shell. The inside posterior mold half is separated from the outside posterior mold half.

The anterior half shell and the posterior half shell are aligned such that circumferential edge surfaces of each of the anterior half shell and the posterior half shell are aligned. The anterior half shell is cured to the posterior half shell at the circumferential edge surfaces to form an AIOL lens shell.

The AIOL lens shell is filled with a liquid polymer at a filler location on the AIOL lens shell. The filler location is sealed on the filled AIOL lens shell. The sealed, filled AIOL lens shell is trimmed to form the AIOL.

Each of the first polymer and the second polymer may include a silicone polymer or an acrylate polymer. The first polymer and the second polymer may be the same polymer. The first polymer and the second polymer may be different polymers.

Aligning the anterior half shell and the posterior half shell may include aligning the outside anterior mold half containing the molded anterior half shell to the outside posterior mold half containing the molded posterior half shell such that circumferential edge surfaces of each of the molded anterior half shell and the molded posterior half shell are aligned.

Bonding the anterior half shell to the posterior half shell at the circumferential edge surfaces to form the AIOL lens shell may include applying a layer of liquid silicon rubber (LSR) or other adhesive polymer to the circumferential edge surface of at least one of the anterior half shell or the posterior half shell. The liquid polymer used to fill the lens may include a bottle-brush polymer, silicone oil, or other material that is optically clear, has an acceptable refractive index, provides the specific mechanical properties to allow shape change, and is safe, biocompatible, and biostable.

In embodiments, the molding may include injecting a polymer into the anterior half shell mold and the posterior half shell mold, and the filler may include injecting a liquid polymer into the AIOL lens shell.

The polymer shell may include a silicone or an acrylate. The liquid polymer filler material may include a bottle-brush polymer, silicone oil, or other type of material. The bottle brush polymer may be optically clear and biostable.

The anterior half shell mold and the posterior half shell mold may be steel tools or other types of metals. The inside surface of a steel anterior half shell mold and the inside surface of a steel posterior half shell mold may be polished to fine surface. The optical surface of the molds may be plated with Nickel. The steel molds may be used to produce polymer molds that are then used to mold the shells.

The anterior half shell may include a flange portion. The anterior half shell flange portion may have an inside surface contiguous with the inside surface of the anterior half shell. The posterior half shell may include a flange portion. The posterior half shell flange portion may have an inside surface contiguous with the inside surface of the posterior half shell.

Prior to the bonding, a layer of liquid silicon rubber (LSR) may be applied to at least one of the inside surface of the anterior half shell flange portion or the inside surface of the posterior half shell flange portion.

Curing the inside surface of the anterior half shell flange portion to the inside surface of the posterior half shell flange portion may produce a micro protrusion of a portion of the anterior half shell flange portion cured to a portion of the posterior half shell flange portion. Trimming may include cutting off the micro protrusion.

Filler the AIOL lens shell may be performed through an opening in the AIOL lens shell. Filler the AIOL lens shell may be performed by puncturing a flange trim or puncturing an area outside the 5 mm optic zone on the anterior or posterior shell. Filler the AIOL lens shell may be performed through a one-way valve in the shell.

The curing temperature, time, pressure and other process conditions will vary depending on the material used.

In accordance with another aspect of the disclosure, a method of manufacturing a single piece (or molded lens) accommodating intraocular lens (AIOL) includes positioning a core with a rib in a first cavity of a mold assembly, aligning a second cavity of the mold assembly with the first cavity, adding uncured shell material to the first cavity of the mold assembly having the core removed (manually removed or dissolved).

The method further includes bonding a preassembled valve in place, curing the uncured shell material inside of the mold assembly to form a cured AIOL, and inserting a sharpened fill cannula into the preassembled check valve.

The method further includes filling the cured AIOL through the cannula, removing the cannula, bonding a distal end of the check valve, curing the distal end of the check valve to form the AIOL, and trimming excess material from the filled AIOL.

Removing the core from the mold assembly may include dissolving the core. Alternatively, removing the core from the mold assembly may include physically extracting the core from the mold assembly.

### EXAMPLES

Examples 1-3 herein provide specific exemplary embodiments of AIOLs having a lens body comprising a shell and filler material with various anterior and posterior shell wall surface configurations, including structural view of the lens body, relevant attributes, and Tabular data illustrating the relationships between power and various properties of the anterior and posterior wall defining the lens body. Example 4 herein provides a specific exemplary embodiments of an AIOLs having a lens body comprising just a flexible, self-supporting material with various anterior and posterior shell wall surface configurations. In embodiments, including the examples, the spherical portions of the lens body surfaces are for providing optical power. Transitions or discontinuities in the curvature of an optical surface, e.g. discontinuity 20 which is circular in most embodiments, represent the border of a spherical surface 32 with a non-spherical surface 34, including the areas in the equator region 27 of the lens body represented by spline 37 whose function is not primarily for providing optical power. In embodiments, the spline or non-spherical surface of the lens body may extend from the spherical transition point on one of the anterior or posterior surfaces to the spherical transition point on the other of the anterior or posterior surfaces, whether interior or exterior. Further, in embodiments, the transition from a spherical surface to a non-spherical surface may occur on only one surface a the shell wall, as in Example 3 wherein the interior posterior surface transitions at multiple locations compared to the exterior posterior surface of the shell wall. In this manner, the curvature of the optical surfaces, e.g. anterior exterior, anterior interior, posterior exterior, posterior interior, as applicable to the respective embodiment, can transition from spherical to non-spherical surfaces, and vice versa, as required to optimize performance of the lens to emulate HCL accommodation.

In the described embodiments, the curvature of an optical surface, e.g. anterior exterior, anterior interior, posterior exterior, posterior interior, as applicable to the respective embodiment, and transition to non-spherical surfaces, e.g. aspheric section 34 or spine 37, can be calculated from changes in radius values as would be understood by those reasonably skilled in the optical arts.

### Example 1

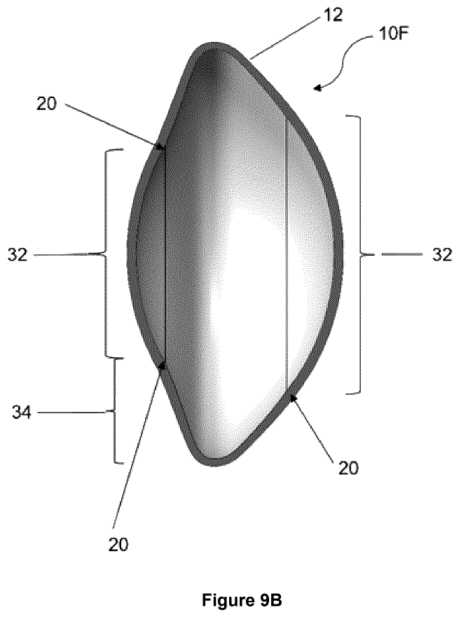

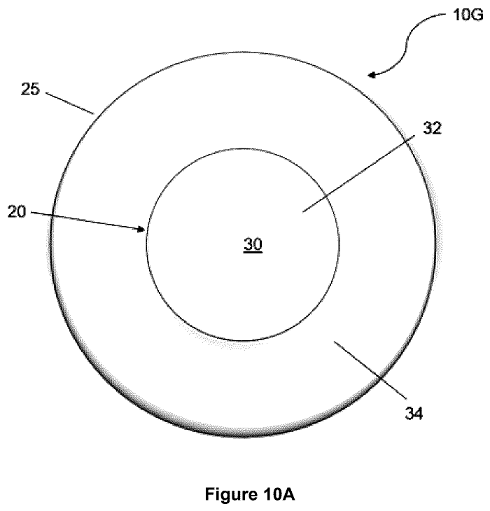

FIGS. 9A and 9B are top and side cut-away views, respectively, of an accommodating intraocular lens 10F comprising a shell and a filler according to an Example 1. In AIOL 10F, the pliable accommodating lens body 15 has a curved exterior anterior surface 16 and a curved exterior posterior surface 18 bounded by perimeter 25, with less than all of one or both of the exterior anterior surface 16 and the exterior posterior surface 18 has a common curvature. As illustrated, the exterior anterior surface 16 is characterized by a discontinuity 20 in optical surface radius within the perimeter 25 of the lens body 15. The external anterior surface pole 24 and location of discontinuity 20 in the external anterior surface 16 are marked accordingly. A central region 30 of the exterior anterior surface 16 within the perimeter 25 comprises a spherical lens portion 32 surrounded by an aspheric lens portion 34. The lens body 15 is capable of accommodation in response to ciliary muscle stimulus without further structure coupled exteriorly of the perimeter 25 of the lens body 15, e.g. haptic structures such as springs, wings, pontoons, auxiliary reservoirs, or other structures used to maintain the lens body within the capsular bag, etc. The structural attributes of AIOL 10F are the listed in Table 5.

TABLE 5

| Attribute | Value (mm) |
|---|---|
| tₐ | 0.2 |
| tₚ | 0.2 |
| R₁ | 5.0 |
| R₂ | 4.8 |
| R₃ | 4.8 |
| R₄ | 5.0 |
| D | 9.2 |
| T | 4.7 |

Table 6 describes the relationship of Power, D, to the radii values of the inside and outside anterior walls, 17 and 16, respectively, and the inside and outside posterior walls 19 and 18, respectively, comprising shell 12 of the AIOL 10F.

### Example 2

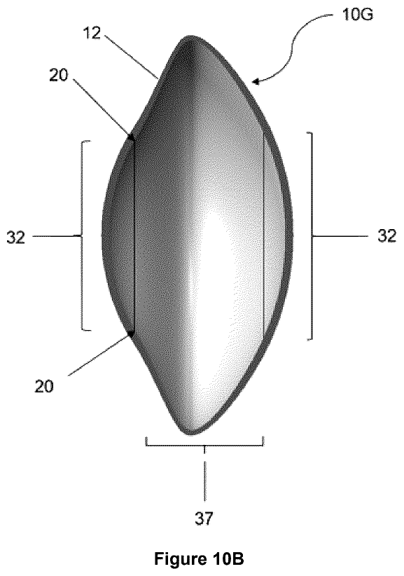

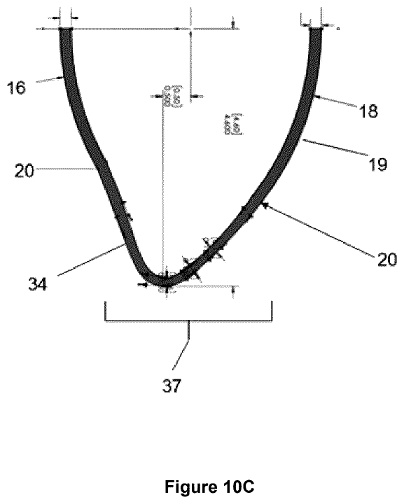

FIGS. 10A, 10B and 10C are top, side cut-away and partial views of an accommodating intraocular lens 10G having a lens body 15 comprising a shell 12 and a filler 14 according to an Example 2. In AIOL 10G, the pliable accommodating lens body 15 has a curved exterior anterior surface 16 and a curved exterior posterior surface 18 bounded by a perimeter 25. Again, less than all of the exterior anterior surface 16 or the exterior posterior surface 18 has a common optical surface curvature. As illustrated, the exterior anterior surface 16 is characterized by a discontinuity 20 in optical surface radius within the perimeter 25 of the lens body 15. The external anterior the surface pole 24 and location of discontinuity 20 in the curvature of the external anterior surface 16 are marked accordingly. A central region 30 of the exterior anterior surface 16 within the perimeter 25 comprises a spherical lens portion 32 surrounded by an aspheric lens portion 34. The lens body 15 is capable of accommodation in response to ciliary muscle stimulus without further structure coupled to the perimeter 25 or equator 27 or other part of the lens body 15.

FIG. 10C shows a side cut way portion of shell 15 the illustrating the relationships of the anterior wall and posterior wall internal and external surfaces to the equator 27, anterior and posterior poles 24 and 23, respectively, the location of disruption 20, and the general configuration of the curved posterior wall and spherical lens portion 32 of the anterior wall to the interconnecting spline section 37 proximate the perimeter 25 of the lens body 15. The structural attributes of AIOL 10G are the listed in Tables 6 and 7.

TABLE 6

| Attribute | Value (mm) |
|---|---|
| ta | 0.2 |
| tp | 0.2 |
| R₁ | 5.0 |
| R₂ | 4.8 |
| R₃ | 6.3 |
| R₄ | 6.5 |
| D | 10.0 |
| T | 4.8 |

TABLE 7

| Attribute | Nominal | Min | Max |
|---|---|---|---|
| Overall Diameter (mm) | 9.2 | 9.1 | 9.3 |
| Central Optic Thickness | 4.7 | 4.6 | 4.8 |
| (mm) | | | |
| Anterior Radius (mm) | 5.0 | 4.9 | 5.1 |
| Posterior Radius (mm) | 5.0 | 4.9 | 5.1 |
| Anterior Shell Thickness | 200 | 170 | 230 |
| (microns) | | | |
| Posterior Shell Thickness | 200 | 70 | 130 |
| (microns) | | | |
| Shell Thickness @ Equator | 150 | 120 | 180 |
| (microns) | | | |

Table 8 describes the relationship of Power, D, to the radii values of the inside and outside anterior walls, 17 and 16, respectively, and the inside and outside posterior walls 19 and 18, respectively, comprising shell 12 of the AIOL 10G.

TABLE 8
Examples 1 and 2

| | |
|---|---|
| Anterior Radius (mm) | Posterior Radius (mm) |

| Power, D | Outside | Inside | Inside | Outside |
|---|---|---|---|---|
| 30.0 | 3.90 | 3.70 | 4.95 | 5.15 |
| 29.5 | 3.90 | 3.70 | 5.15 | 5.35 |
| 29.0 | 3.90 | 3.70 | 5.40 | 5.60 |
| 28.5 | 3.90 | 3.70 | 5.70 | 5.90 |
| 28.0 | 3.90 | 3.70 | 5.90 | 6.10 |
| 27.5 | 3.90 | 3.70 | 6.30 | 6.50 |
| 27.0 | 5.00 | 4.80 | 4.80 | 5.00 |
| 26.5 | 5.00 | 4.80 | 4.95 | 5.15 |
| 26.0 | 5.00 | 4.80 | 5.15 | 5.35 |
| 25.5 | 5.00 | 4.80 | 5.40 | 5.60 |
| 25.0 | 5.00 | 4.80 | 5.70 | 5.90 |
| 24.5 | 5.00 | 4.80 | 5.90 | 6.10 |
| 24.0 | 5.00 | 4.80 | 6.30 | 6.50 |
| 23.5 | 6.50 | 6.30 | 4.95 | 5.15 |
| 23.0 | 6.50 | 6.30 | 5.15 | 5.35 |
| 22.5 | 6.50 | 6.30 | 5.40 | 5.60 |
| 22.0 | 6.50 | 6.30 | 5.70 | 5.90 |
| 21.5 | 6.50 | 6.30 | 5.90 | 6.10 |
| 21.0 | 6.50 | 6.30 | 6.30 | 6.50 |
| 20.5 | 9.00 | 8.80 | 4.95 | 5.15 |
| 20.0 | 9.00 | 8.80 | 5.15 | 5.35 |
| 19.5 | 9.00 | 8.80 | 5.40 | 5.60 |
| 19.0 | 9.00 | 8.80 | 5.70 | 5.90 |
| 18.5 | 9.00 | 8.80 | 5.90 | 6.10 |
| 18.0 | 9.00 | 8.80 | 6.30 | 6.50 |

### Example 3

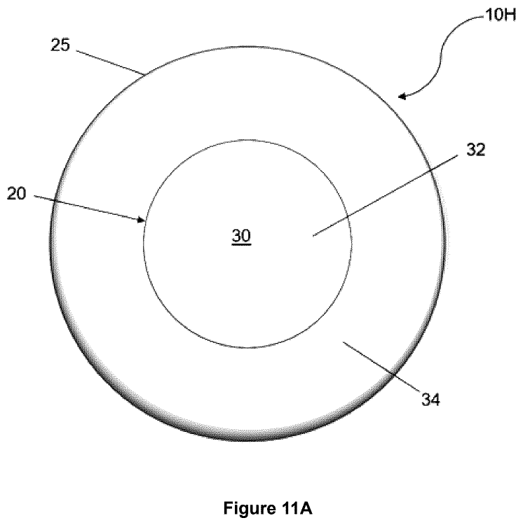

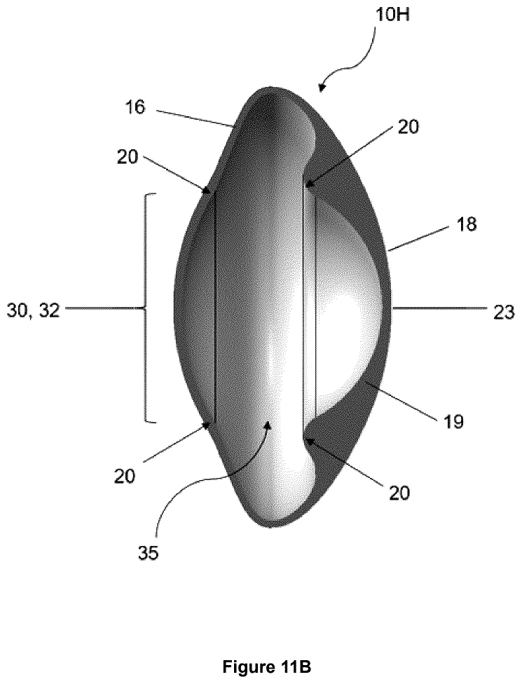

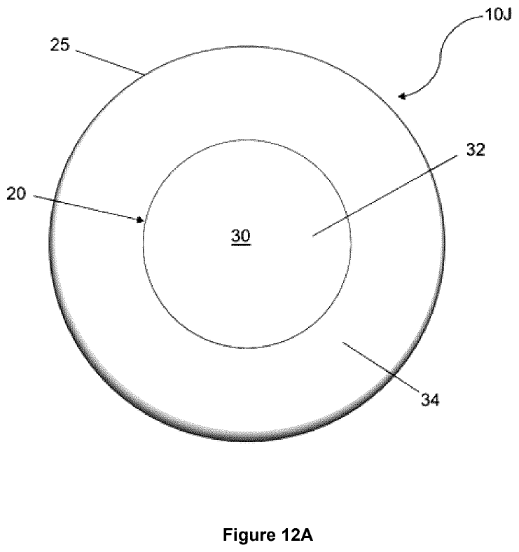

FIGS. 11A and 11B are top and side cut-away views, respectively, of an accommodating intraocular lens 10H having a lens body 15 comprising shell 14 and a filler 14 according to an Example 3. In AIOL 10H, the pliable lens body 15 has an interior volume 35 defined by an interior anterior surface 17 and an interior posterior surface 19 bounded by perimeter 20, wherein one or both of the interior anterior wall surface 17 and the interior posterior wall surface 19 is characterized by a discontinuity 20 in the interior volume surface within the perimeter 20 of the lens body 15. In the illustrative embodiment, the interior posterior wall surface 19 is characterized by multiple abrupt changes in surface radius within the interior volume 35. The external posterior surface pole 23 and location of discontinuity 20 in the curvature of the internal posterior surface 19 are marked accordingly. The structural attributes of AIOL 10H are the listed in Table 9.

TABLE 9

| Attribute | Value (mm) |
|---|---|
| ta | 0.2 |
| tp | 0.2-0.5 |
| R₁ | 5.0 |
| R₂ | 4.8 |
| R₃ | 2.7 |
| R₄ | 6.0 |
| D | 9.5 |
| T | 4.7 |

Table 10 describes the relationship of Power, D, to the radii values of the inside and outside anterior walls, 17 and 16, respectively, and the inside and outside posterior walls 19 and 18, respectively, comprising shell 12 the AIOL 10H.

TABLE 10
Example 3

| | |
|---|---|
| Anterior Radius (mm) | Posterior Radius (mm) |

| Power, D | Outside | Inside | Inside | Outside |
|---|---|---|---|---|
| 30.0 | 4.50 | 4.30 | 4.30 | 4.50 |
| 29.5 | 4.50 | 4.30 | 4.00 | 4.50 |
| 29.0 | 4.50 | 4.30 | 3.60 | 4.50 |
| 28.5 | 4.50 | 4.30 | 3.35 | 4.50 |
| 28.0 | 4.50 | 4.30 | 3.10 | 4.50 |
| 27.5 | 4.50 | 4.30 | 2.85 | 4.50 |
| 27.0 | 5.00 | 4.80 | 4.80 | 5.00 |
| 26.5 | 5.00 | 4.80 | 4.40 | 5.00 |
| 26.0 | 5.00 | 4.80 | 3.95 | 5.00 |
| 25.5 | 5.00 | 4.80 | 3.60 | 5.00 |
| 25.0 | 5.00 | 4.80 | 3.20 | 5.00 |
| 24.5 | 5.00 | 4.80 | 5.40 | 6.00 |
| 24.0 | 5.00 | 4.80 | 4.70 | 6.00 |
| 23.5 | 5.00 | 4.80 | 4.30 | 6.00 |
| 23.0 | 5.00 | 4.80 | 3.80 | 6.00 |
| 22.5 | 5.00 | 4.80 | 3.50 | 6.00 |
| 22.0 | 5.00 | 4.80 | 3.20 | 6.00 |
| 21.5 | 5.00 | 4.80 | 2.95 | 6.00 |
| 21.0 | 5.00 | 4.80 | 2.70 | 6.00 |
| 20.5 | 5.50 | 5.30 | 4.50 | 7.00 |
| 20.0 | 5.50 | 5.30 | 4.00 | 7.00 |
| 19.5 | 5.50 | 5.30 | 3.65 | 7.00 |
| 19.0 | 5.50 | 5.30 | 3.30 | 7.00 |
| 18.5 | 5.50 | 5.30 | 3.05 | 7.00 |
| 18.0 | 5.50 | 5.30 | 2.80 | 7.00 |

### Example 4

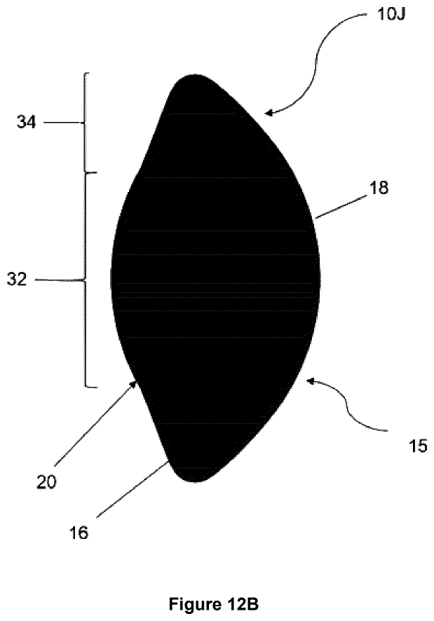

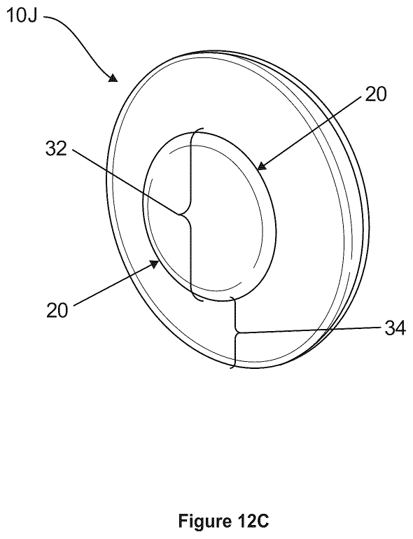

FIGS. 12A-12C are top, side cut-away, and perspective views, respectively, of an accommodating intraocular lens 10J comprising only a flexible lens body 15, without a shell, according to an Example 4. In AIOL 10J, the pliable lens body 15 comprises an optically clear material, with the lens body 15 defining an exterior anterior surface 16 and an exterior posterior surface 18 bounded by perimeter 20. One or both of the exterior anterior surface 16 and the exterior posterior surface 18 is characterized by a discontinuity 20 in the curvature of the respective exterior surface within the perimeter 20 of the lens body 15. The external anterior surface pole 24 and location of discontinuity 20 in the curvature of the external anterior surface 16 are marked accordingly. In embodiments, the optically clear material comprises an optically clear copolymer or a bottle brush polymer or bottle brush copolymer. The structural attributes of AIOL 10J are the listed in Table 11.

TABLE 11

| Properties | Typical Value |
|---|---|
| Appearance | Clear |
| Refractive Index | 1.350-1.550 |
| Specific Gravity | 1.0-1.3 |
| Hardness, Shore A | 6-12 |
| Tensile Strength, psi | 500-1000 |
| Elongation, % | 700-1500 |
| Modulus, 100%, psi | 1-50 |
| Anterior Radius | Varies according to |
| (mm) R₁ | desired lens power |
| Posterior Radius | Varies according to |
| (mm) R₂ | desired lens power |
| Overall Diameter | 8.5-11.0 |
| (mm), D | |
| Central Optic | 3.5-5.5 |
| Thickness (mm), | |
| T | |

Table 12 describes the relationship of Power, D, to the radii values of the outside anterior wall 16 and outside posterior wall 18 comprising the lens body 15 of the AIOL 10J.

TABLE 12
Example 4

| Power, D | Anterior Radius (mm) | Posterior Radius (mm) |
|---|---|---|
| 30.0 | 4.00 | 5.20 |
| 29.5 | 4.00 | 5.40 |
| 29.0 | 4.00 | 5.60 |
| 28.5 | 4.00 | 5.80 |
| 28.0 | 4.00 | 6.10 |
| 27.5 | 4.00 | 6.40 |
| 27.0 | 4.50 | 5.80 |
| 26.5 | 4.50 | 6.10 |
| 26.0 | 4.50 | 6.40 |
| 25.5 | 5.00 | 5.80 |
| 25.0 | 5.00 | 6.10 |
| 24.5 | 5.00 | 6.40 |
| 24.0 | 5.00 | 6.70 |
| 23.5 | 5.80 | 5.80 |
| 23.0 | 5.80 | 6.10 |
| 22.5 | 5.80 | 6.40 |
| 22.0 | 5.80 | 6.70 |
| 21.5 | 5.80 | 7.00 |
| 21.0 | 5.80 | 7.40 |
| 20.5 | 5.80 | 7.90 |
| 20.0 | 5.80 | 8.30 |
| 19.5 | 7.00 | 7.00 |
| 19.0 | 7.00 | 7.40 |
| 18.5 | 7.00 | 7.90 |
| 18.0 | 7.00 | 8.30 |

The reader will appreciate that the AIOL embodiments disclosed herein are capable of addressing specific visual conditions through modification of the curvature of the anterior or posterior wall surfaces, as applicable, to provide an amount of accommodation to allow a continuous range of vision that imitates the healthy human crystalline lens, all without further structure coupled to the perimeter of the lens body. Further, the disclosed AIOL attributes are selected to maximize accommodation range when subjected to ocular physiological conditions. Any of the four optical surfaces, e.g. anterior wall exterior surface, anterior wall interior surface, posterior wall exterior surface, and posterior wall interior surface, can be modified to enhance overall optical performance to extend the overall depth of focus of the disclosed AIOL or for correction of specific conditions such as correction for corneal astigmatism, or correction for corneal spherical aberration, etc. Once assembled, the AIOL is compatible with existing or novel intraocular lens delivery procedures and delivery system, developed for delivery via small incision implantation into the eye.

The embodiments described above are intended to be merely exemplary; numerous variations and modifications will be apparent to those skilled in the art including, potentially, alternative geometries (e.g., to address different ocular conditions, eye shapes, eye sizes, etc.), alternative materials for the shell and/or the filler, etc. Such variations and modifications are intended to be within the scope of the present invention as defined by any of the appended claims. Any references to “invention” or “embodiments” are intended to be exemplary and should not be construed to refer to all possible embodiments unless the context otherwise requires. Accordingly, the described embodiments are to be considered in all respects only as illustrative and not restrictive.

At various places in the present specification, values are disclosed in groups or in ranges. It is specifically intended that the description includes each and every individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

For purposes of clarity and a concise description, features are described herein as part of the same or separate embodiments, however, it will be appreciated that scope of the concepts may include embodiments having combinations of all or some of the features described herein.

It will be apparent to those recently skilled in the art that modifications to the apparatus and process disclosed here in may occur, including substitution of various component values or nodes of connection, without parting from the true spirit and scope of the disclosure as defined by the claims set forth herein.

What is claimed is:

1.-25. (canceled)

26. An accommodating intraocular lens device comprising:
a pliable lens body having an interior volume defined by an interior anterior surface and an interior posterior surface bounded by a perimeter, wherein one of the interior anterior surface and the interior posterior surface is characterized by a discontinuity in curvature within the interior volume of the lens body.

27. The accommodating intraocular lens of claim 26, wherein the interior posterior surface is characterized by an abrupt change in curvature within the interior volume.

28. The accommodating intraocular lens of claim 27, wherein the abrupt change in curvature of the interior posterior surface comprises an increase of greater than approximately 5%.

29. The accommodating intraocular lens of claim 27, wherein the abrupt change in curvature of the interior posterior surface comprises a decrease of greater than approximately 5%.

30. The accommodating intraocular lens of claim 26, wherein the interior anterior surface is characterized by an abrupt change in curvature within the interior volume.

31. The accommodating intraocular lens of claim 30, wherein the abrupt change in curvature of the interior anterior surface comprises an increase of greater than approximately 5%.

32. The accommodating intraocular lens of claim 30, wherein the abrupt change in curvature of the interior anterior surface comprises a decrease of greater than approximately 5%.

33. An accommodating intraocular lens device comprising:
a pliable accommodating lens body having a curved exterior anterior surface and a curved exterior posterior surface bounded by a perimeter, wherein less than all of the exterior anterior surface or the exterior posterior surface has a common curvature.

34. An accommodating intraocular lens device comprising:
a pliable lens body having an exterior anterior surface and an exterior posterior surface bounded by a perimeter, wherein one of the exterior anterior surface and exterior posterior surface is characterized by a discontinuity in curvature within the perimeter of the lens body.

35. An accommodating intraocular lens device comprising:
a pliable lens body having an exterior anterior surface and an exterior posterior surface bounded by a perimeter, wherein one of the interior anterior surface and interior posterior surface is characterized by a discontinuity in curvature within the perimeter of the lens body.

36.-42. (canceled)

43. The accommodating intraocular lens of claim 26, wherein the discontinuity in curvature within the interior volume of the lens body comprises a transition from a spherical interior posterior surface to a non-spherical interior posterior surface.

44. The accommodating intraocular lens of claim 26, wherein the discontinuity in curvature within the interior volume of the lens body comprises a transition from a spherical interior anterior surface to a non-spherical interior anterior surface.

45. The accommodating intraocular lens of claim 34, wherein the discontinuity in curvature within the perimeter of the lens body comprises a transition from a spherical exterior posterior surface to a non-spherical exterior posterior surface.

46. The accommodating intraocular lens of claim 34, wherein the discontinuity in curvature within the perimeter of the lens body comprises a transition from a spherical exterior anterior surface to a non-spherical exterior anterior surface.

47.-56. (canceled)

57. The accommodating intraocular lens of claim 35, wherein the interior posterior surface is characterized by an abrupt change in curvature.

58. The accommodating intraocular lens of claim 57, wherein the abrupt change in curvature of the interior posterior surface comprises an increase of greater than approximately 5%.

59. The accommodating intraocular lens of claim 57, wherein the abrupt change in curvature of the interior posterior surface comprises a decrease of greater than approximately 5%.

60. The accommodating intraocular lens of claim 35, wherein the interior anterior surface is characterized by an abrupt change in curvature.

61. The accommodating intraocular lens of claim 60, wherein the abrupt change in curvature of the interior anterior surface comprises an increase of greater than approximately 5%.

62. The accommodating intraocular lens of claim 60, wherein the abrupt change in curvature of the interior anterior surface comprises a decrease of greater than approximately 5%.
